(19) United States
(12) Patent Application Publication
(10) Pub. No.: US 2003/0040507 A1
(43) Pub. Date: Feb. 27, 2003

(54) Pharmaceutical composition comprising ifosfamide and carnitine

(76) Inventors: Bernd Nickel, Muhltal (DE); Joerg Pohl, Dietzenbach (DE); Thomas Nolte, Borgholzhaussen (DE); Peter Hilgard, Frankfurt (DE); Jurgen Engel, Alzenau (DE); J. S. Schlenzig, Frankfurt (DE)

Correspondence Address: PILLSBURY WINTHROP, LLP, P.O. BOX 10500, MCLEAN, VA, 22102, US

(21) Appl. No.: 10/266,708

(22) Filed: Oct. 9, 2002

Related U.S. Application Data
(63) Continuation of application No. 09/127,550, filed on Aug. 3, 1998, now abandoned.

(30) Foreign Application Priority Data
Aug. 1, 1997 (DE) 197 33 305.2

(51) Int. Cl. A61K 31/66; A61K 31/195

(52) U.S. Cl. 514/110; 514/561

(57) ABSTRACT

The invention relates to the use of a combination of ifosfamide and carnitine, in particular L-carnitine, for the production of tumour pharmaceuticals having decreased side effects. The results show clearly that the side effect produced by ifosfamide (damage to the proximal tubule of the kidney) is antagonized in animals by L-carnitine. It was furthermore possible to show that the antitumour action of ifosfamide is not affected in combination with L-carnitine. The combination also caused no new side effects in the animals.

The invention relates to novel pharmaceutical compositions for use for cancer therapy, comprising ifosfamide and carnitine or its derivatives, having improved tolerability, in particular lower nephrotoxicity.

It is known and described that ifosfamide causes side effects in patients in the treatment of cancer. These are manifested in the ifosfamide-treated patients by damage to the proximal tubule of the kidney. (Pediatr. Nephrol. 1994, 8:157-163; Renal Physiol. Biochem. 1992 15:289-301 ibid. 1993, 16:285-298) Furthermore, the coadministration of oxaphosphorinanes such as ifosfamide with mercaptoethanesulphonate (Mesna) for cancer treatment follows from various publications, the urotoxic action being lowered.

It is known of carnitine that it is employed in systemic carnitine deficiency and muscular dystrophy with lipid accumulation. It furthermore improves the load capacity and the regenerability of the muscle (Múnch. med. Wschr. 138(1996) No. 23 p. 53

It was possible in world-wide studies to achieve an improvement by means of L-carnitine in neurological attacks and brain function disorders such as senile dementia and Alzheimer's disease.

It is further employed in the area of cardiovascular diseases in the treatment of acute and chronic myocardial ischaemia, angina pectoris and also cardiac arrhythmia and insufficiency as well as in chronic uraemia in dialysis patients.

EP 0 722 724 discloses the use of L-carnitine and its derivatives for decreasing the toxic effect of cyclosporin A and other immunosuppressants.

Finally, experiments on rats are known from a study by Schlenzig et al. in Eur. J. Pediatr. (1995) 154:686-690 to administer L-carnitine together with ifosfamide, an improvement in the clinical result (decreased lethargy) being observed and only a slight lowering of the intermediates of the tricarboxylic acid cycle in the urine. It is pointed out that the addition of L-carnitine could offer an improvement in the ifosfamide treatment.

The aim of the present invention was to characterize substances which, in combination with ifosfamide, antagonize the known side effects (damage to the proximal tubule of the kidney). It must be ensured in this case that the antitumour action of ifosfamide is not abolished or weakened by combination with the antidote and no additional side effects occur due to the administration of the combination.

In accordance with the set object, comparative investigations were investigated [sic] out with respect to effect on the nephrotoxicity in healthy and tumour-bearing rats after administration of ifosfamide on its own and in combination with L-carnitine. The doses for L-carnitine were selected such that the compound itself causes no side effects at all in the animals. On administration of ifosfamide on its own, as was to be expected, clear dose-dependent damage to the proximal tubule of the kidneys occurs in both animal groups.

This specific damage is surprisingly antagonized significantly according to the invention by the simultaneous administration of L-carnitine (2×100 mg/kg i.v.). (Tab. 1).

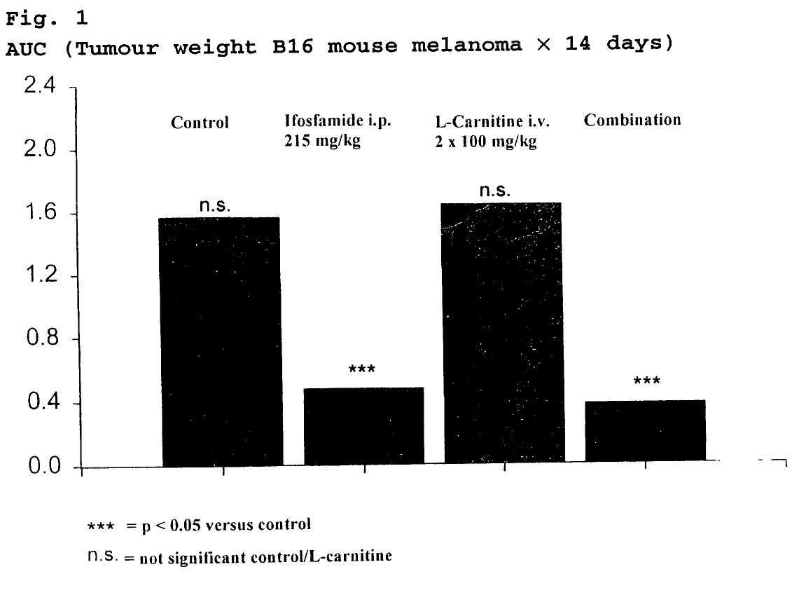

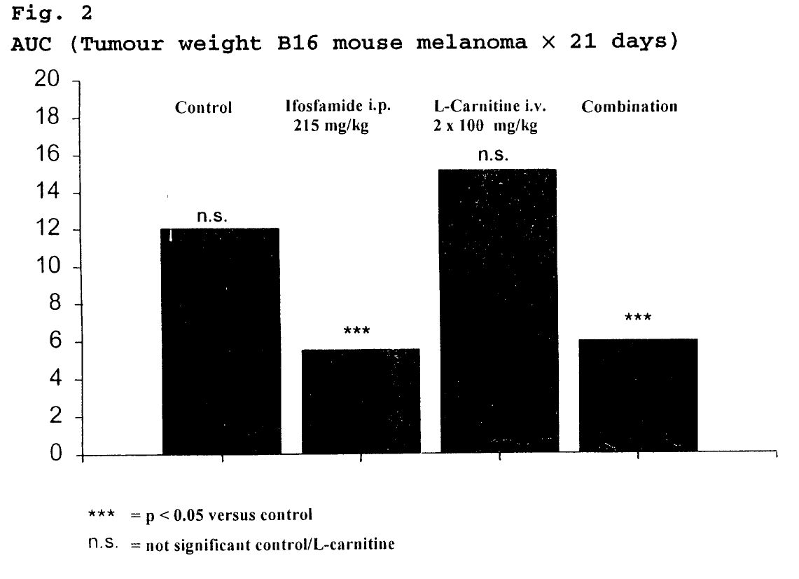

The antitumour action of ifosfamide is not affected in combination with L-carnitine (FIGS. 1 and 2).

The invention consequently relates to the use of L-carnitine in free base form or as a physiologically tolerable salt such as L-tartrate, magnesium citrate or as acetyl-L-carnitine HCl for the production of cytostatics with ifosfamide, to be precise in fixed or free combination. The doses administered in this case are in the known orders of magnitude, i.e. in the case of carnitine or its derivative up to 5 g can be administered per patient with the ifosfamide dose. However a ratio of ifosfamide to carnitine from 1:10 to 1:20 is preferred.

Furthermore, to avoid the known toxic action of ifosfamide on the bladder, Mesna can additionally be employed in the same or as a separate dose unit in the dose known per se (Tab. 2). A complex protection of the kidney and the bladder during tumour treatment with ifosfamide is thus achieved.

Patent claims

1. Pharmaceutical composition for use in tumour therapy, comprising ifosfamide and carnitine

2. Use of a pharmaceutical composition consisting of ifosfamide and carnitine for the production of a medicament for the treatment of oncoses.

3. Use of a pharmaceutical composition consisting of ifosfamide and carnitine for the treatment of oncoses.

4. Pharmaceutical composition according to claims 1-3, the carnitine being present as L-carnitine base or, in the form of physiologically tolerable salts, as L-tartrate, magnesium citrate or as acetylcarnitine hydrochloride.

5. Pharmaceutical composition according to claim 1, characterized in that both the ifosfamide and the carnitine is [sic] present and used in the form of injection solutions.

6. Use according to claims 2 and 3, characterized in that L-carnitine is administered together with or separately from the ifosfamide administration in the form of drinking solutions or as an injection or infusion.

7. Pharmaceutical composition according to claim 1, characterized in that the weight ratio of the constituents ifosfamide to carnitine is 1:10 to 1:20.

8. Procedure for the treatment of oncoses, characterized in that a therapeutic dose of ifosfamide is administered together with L-carnitine, either in one dose unit or in separate dose units

9. Pharmaceutical composition according to claims 1, 4, 5 and 7, characterized in that, in addition to ifosfamide and L-carnitine, it also contains Mesna.

10. Procedure for treatment of oncoses, characterized in that a therapeutic dose of ifosfamide is administered either in one dose unit or in separate dose units together with L-carnitine and Mesna.
